(19) United States
(12) Patent Application Publication
(10) Pub. No.: US 2023/0295708 A1
(43) Pub. Date: Sep. 21, 2023

(54) REAL-TIME POLYMERASE CHAIN REACTION (PCR) SYSTEM FOR POINT-OF-CARE MEDICAL DIAGNOSIS

(71) Applicant: University of Dayton Research Institute, Dayton, OH (US)

(72) Inventors: Vamsy Chodavarapu, Dayton, OH (US); Tchamie Kadja, Dayton, OH (US)

(21) Appl. No.: 18/185,001

(22) Filed: Mar. 16, 2023

Related U.S. Application Data
(60) Provisional application No. 63/320,517, filed on Mar. 16, 2022.

(51) Int. Cl. C12Q 1/686; B01L 3/00; B01L 7/00

(52) U.S. Cl. CPC C12Q 1/686; B01L 3/502715; B01L 7/52; B01L 2300/025; B01L 2300/1827; B01L 2300/0609; B01L 2300/0663

(57) ABSTRACT

A real-time polymerase chain reaction (PCR) system includes a thermal cycler that controls a temperature of the system, a control system that controls the thermal cycler, an optical system and readout that displays results of a test of the sample for pathogens, and a network device that conveys the results of the test of the sample for pathogens. The thermal cycler comprises a denaturation controller that controls temperature of a denaturation phase, an annealing controller that controls temperature of an annealing phase, and an extension controller that controls temperature of an extension phase, where the three controllers are each individually selected by the control system.

### CROSS REFERENCE TO RELATED APPLICATIONS

This application claims the benefit of U.S. Provisional Patent Application Ser. No. 63/320,517, filed Mar. 16, 2022, entitled REAL-TIME POLYMERASE CHAIN REACTION (PCR) SYSTEM FOR POINT-OF-CARE MEDICAL DIAGNOSIS, the disclosure of which is hereby incorporated by reference.

### BACKGROUND

Various aspects of the present invention relate generally to polymerase chain reaction (PCR) systems for point of care medical diagnoses and specifically to low cost, real-time polymerase chain reaction (PCR) systems for point of care medical diagnoses.

Recent global health crises due to the prevailing Coronavirus Disease 2019 (COVID-19) pandemic has placed significant strain on health care facilities such as hospitals and clinics around the world. In addition to critical shortage of healthcare personnel, ventilators and personal protective equipment, there is a widespread need for diagnostic tools to quickly assess the patient health. Polymerase Chain Reaction (PCR) system is a widely used diagnostic tool to test COVID-19 patient samples. The social and economic difficulties being faced in getting the pandemic under control is causing the rise of new Severe Acute Respiratory Syndrome Coronavirus 2 (SARS-CoV-2) variants that continue to extend the duration of the pandemic.

### BRIEF SUMMARY

According to aspects of the present invention a low cost, real-time polymerase chain reaction (PCR) system includes a thermal cycler that controls a temperature of the system, a control system that controls the thermal cycler, an optical system and readout that displays results of a test of the sample for pathogens, and a network device that conveys the results of the test of the sample for pathogens. The thermal cycler comprises a denaturation controller that controls temperature of a denaturation phase, an annealing controller that controls temperature of an annealing phase, and an extension controller that controls temperature of an extension phase, where the three controllers are each individually selected by the control system.

### BRIEF DESCRIPTION OF THE SEVERAL VIEWS OF THE DRAWINGS

FIG. 1 a block diagram illustrating structure of an embodiment of the real-time polymerase chain reaction (PCR) system, according to aspects of the present disclosure;

FIG. 2 is a thermal flowchart between temperature cycling objects of a real-time polymerase chain reaction system where blue illustrates cooling stages and red heating stages, according to aspects of the present disclosure;

FIG. 3 is a schematic illustrating an optical system of the real-time polymerase chain reaction system, according to aspects of the present disclosure;

FIG. 4 is a block diagram illustrating structure of an embodiment of the real-time polymerase chain reaction (PCR) system illustrating a power supply, thermal control, an optical subsystem, a control unit, and a communication module of the embodiment of the PCR system, according to various aspects of the present disclosure;

FIG. 5 is a graph illustrating resistance-temperature characteristics of an embodiment of the PCR system, according to various aspects of the present disclosure;

FIG. 6 is a block diagram illustrating a conventional proportional-integral-derivative (PID) controller;

FIG. 7 is a block diagram illustrating a proportional-integral-derivative temperature controller with individual PID controllers for different aspects of temperature control, according to various aspects of the present disclosure;

FIG. 8 is a chart illustrating temperature over time of an embodiment of the PCR system, according to various aspects of the present disclosure;

FIG. 9 illustrates fluorescence intensity using a StepPlusOne system; and

FIG. 10 illustrates fluorescence intensity using an embodiment of the PCR system, according to aspects of the present disclosure.

### DETAILED DESCRIPTION

Since 2020, the world has been experiencing a severe Coronavirus Disease 2019 (COVID-19) pandemic caused by the Severe Acute Respiratory Syndrome Coronavirus 2 (SARS-CoV-2). According to the World Health Organization (WHO), as of 11 Jan. 2022, there have been 312,173,462 confirmed cases of COVID-19, including 5,501,000 deaths. Across the world, local, state/provincial and central governments are struggling to address the extraordinary human, economic and social tragedy caused by the pandemic. The COVID-19 is the worst pandemic in past 100 years since the Spanish flu, also known as the Great Influenza epidemic or the 1918 influenza pandemic, which was an exceptionally deadly global influenza pandemic caused by the H1N1 influenza A virus. An estimated 300 million people had been infected in four successive waves and with estimates of deaths ranging from 50 million to 100 million. Since the year 2000, the world experienced many pandemics including the Severe Acute Respiratory Syndrome, a viral respiratory illness caused by a coronavirus (SARS-CoV) in 2003, H1N1 Virus (Swine Flu) in 2009, and Zika Virus in 2016. Medical equipment like a Polymerase Chain Reaction (PCR) system have played a crucial role during the COVID-19 pandemic. The PCR test for COVID-19 has been frequently used to analyze the upper respiratory specimens from people, looking for genetic material (ribonucleic acid or RNA) of SARS-CoV-2. The pandemic has caused a dire impact on all countries but emerging and resource poor countries which lack adequate healthcare infrastructure are facing more difficulties. Low-cost and simple-to-use polymerase Chain Reaction (PCR) systems would allow all countries including developed, emerging and resource poor countries to better handle current and future health crises by quickly testing the patient samples.

In addition, research has shown a prevalence in foodborne pathogens worldwide, with numerous deaths due to complications related to bacterial infections. These findings raise the concern for bacterial testing in food and drinking water. PCR is, in this instance as well, a technique used for food and water quality assessment. Forensics employ PCR also for DNA analysis. Many commercially available PCR devices, besides being costly, are bulky and hard to move around. This limits their practicality, in case they should be used “on the move”. Gel electrophoresis is a common technique used to assess amplicon presence after a PCR run has been completed. This requires additional time, additional equipment and reagents and generates extra waste. Fluorescence, in embodiments of the present disclosure, is used as a faster and wasteless alternative, but at a much higher additional cost. Some low-cost PCR system designs exist, but those designs lack customization, being limited to fixed 2-step or 3-step PCR protocol, with fixed steps in temperature and a fixed number of cycles. But real-life DNA assays settings should be tailored accordingly. Many PCR system designs are not portable, being made of discrete components, and above all, many lack the fluorescence capability.

Further, in recent years, foodborne illnesses caused by pathogens such as E. coli 0157, Salmonella, L. monocytogenes, and Campylobacter are affecting large populations causing up to 420,000 deaths yearly of which 30% are children under 5 years old. Drinking water wells used in many parts of the world are also a major source of bacterial contamination. These findings raise the need for real-time and rapid bacterial testing in food and drinking water where PCR testing would be an excellent solution to address those needs.

Since its introduction in the 1980s, PCR has transformed the field of biological sciences and medicine. PCR is an enzymatic assay that allows for the amplification of a specific DNA fragment from a complex pool of DNA. It plays a key role in many fields including pathogen analysis, environmental monitoring, and food inspection. Commercial PCR systems are expensive and large size, with prices from $4,000 for simple PCR machines to more than $90,000 for some real time PCR systems. Various types of low-cost PCR systems have been developed previously. Further, many miniaturized system-on-chip and microfluidics technology have been used to develop PCR systems. Most low-cost designs developed to date have limited fixed 2-step or fixed 3-step temperature setting used for thermal cycling, while others require reprogramming and altering the system hardware configuration if changes in the PCR protocol were to be made. Other designs lack precise temperature control which makes them unsuitable for clinically relevant sensitive and selective reactions. While other low-cost PCR designs do not incorporate real-time reaction monitoring with built-in fluorescence capabilities, which would require the use of time-consuming gel electrophoresis. Real-world assays require flexible user-defined parameters such as number of steps, hold time, step temperature and number of cycles.

A practical, cost-efficient, simple-to-use, portable, and real-time PCR system uses a metallic thermal block and heating lid which may be easily made with 3D printing. Other parts of the system include commercial off-the-shelf items. The complete system is assembled in a custom-made low-cost 3D printed enclosure. Listeria monocytogenes are used as a test sample target to assess an efficiency of the PCR system. Being able to quickly detect listeria Monocytogenes at a low number of amplicons is useful to examine functionality of embodiments of the PCR system described herein. A test sample without any sample pretreatment is analyzed using the developed PCR system.

System Architecture

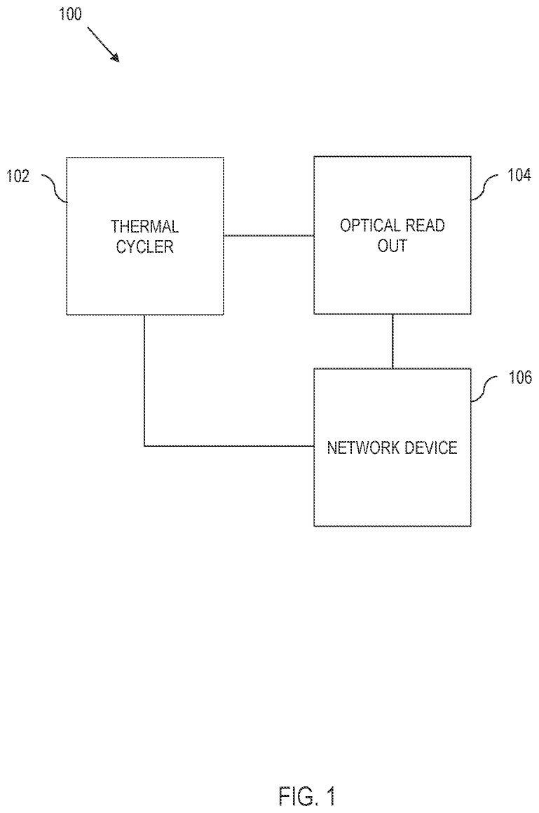

Turning now to the figures, and in particular to FIG. 1, a block diagram of a PCR system 100 is shown. The PCR system has three main parts, a thermal cycler 102, an optical read-out system 104, and a network device 106.

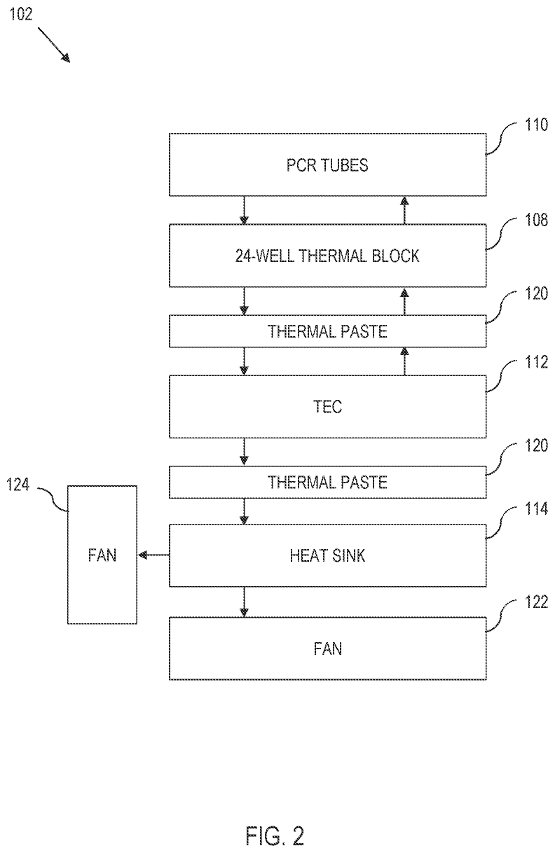

Turning to FIG. 2, a structural diagram of the path of heat flow between various objects of the thermal cycler 102 is shown, the temperature cycler 102 was built using a high-throughput PCR architecture. The parts include a 24-well metallic thermal block 108 incorporating two heaters (e.g., 70.3Ω and 68.7Ω heaters). Further, the system 100 has a 14.7Ω (20° C.)−20.93Ω (105° C.) heated lid. The thermal block 108 holds PCR reaction tubes 110 (e.g., one PCR tube for each well) while serving as a primary heating component. For example, the thermal block may be heated using a 67V, 2A DC power supply. Meanwhile, a cooling process during thermal cycling is achieved using two thermoelectric cooler (TEC) Peltier modules (e.g., CUI Devices, CP105433H, 40 mm×40 mm×3.3 mm) 112 in conjugation with a heat sink (e.g., 139.7 mm×38.1 mm×127 mm, Wakefield-Vette, 394-2AB) 114. The TECs 112 work in junction with the thermal block 108 to heat specimens in the tubes 110 during the high temperature phase of the PCR protocol. The TECs 112 are powered with a 12V, 20A DC power supply, which also serves as a power source for the rest of the system 100. High performance thermal paste (e.g., Corsair XTM50) 120 is used for maximum heat conduction between all thermal cycler objects. Two fans (120 mm×120 mm×25 mm, 12V) 122, 124 were added to the system 100 to keep the heat sink 114 from overheating and to accelerate the cooling process.

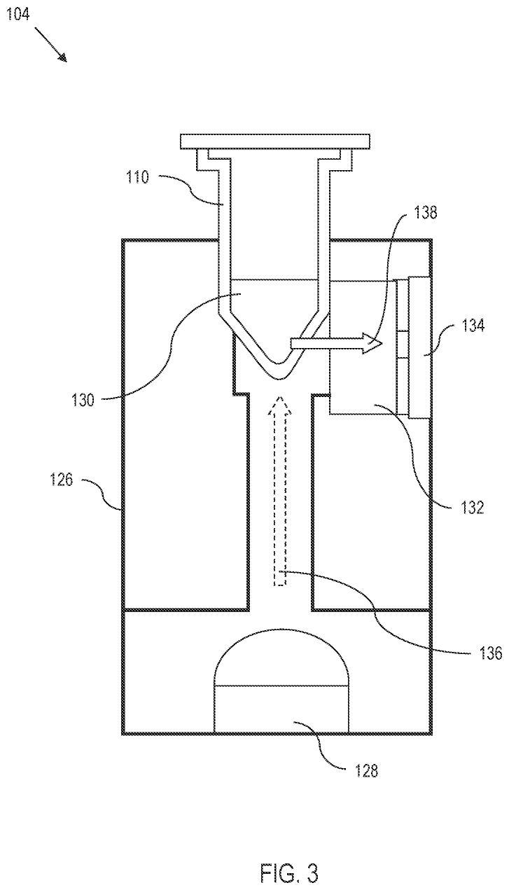

Real-time PCR also known as Quantitative PCR (qPCR) is achieved by the detection of emitted light from an excited fluorophore which is embedded in DNA amplicons during a PCR annealing phase, using a light-emitting diode (LED) as excitation source. As discussed above, using fluorescence allows for real-time DNA assay without relying solely on gel electrophoresis. FIG. 3 illustrates a diagram of the low-cost optical system 100, which includes an LED light source 128, a 3D printed receptacle 126 that can hold a PCR tube 110 with a DNA solution 130, an optical filter 132, and a chip-scale spectrometer 134. In operation, the incoming excitation light 136 travels from underneath the PCR tube 110 while the emitted light 138 is detected at right angle from the excitation spectrum to minimize background noise. The emission wavelength is detected using a low-cost chip-scale 10-channel spectrometer (e.g., AMS AG, model: AS7341) 134 with an orange optical filter (e.g., 546 nm center wavelength, Full Width at Half Maximum (FWHM) of 8 nm) 132 in the foreground. The spectrometer 134 utilizes a light sensor that can detect center wavelengths at 415 nm, 445 nm, 480 nm, 515 nm, 555 nm, 590 nm, 630 nm and 680 nm (all with FWHM of 8 nm), which is useful for different types of fluorescence applications.

Real-time fluorescence detection data from the photodetector is transmitted to a user device or server via the network module 106. For example, a low cost ESP8266 WiFi module may be used as the network module. This setting makes it compatible with most of the current WiFi compatible devices such as phones, computers, and tablets.

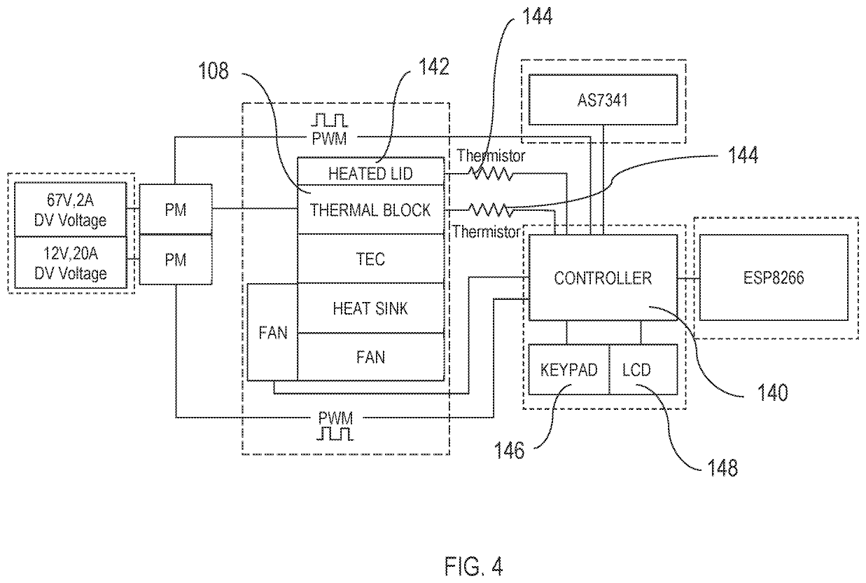

Turning now to FIG. 4, in embodiments of the PCR system 100, temperature tracking, heating, cooling and data transmission is performed under the control of a microcontroller (e.g., low-cost 8-bit RISC-based ATmega2560) 140. The microcontroller 140 incorporates a multichannel 10-bit Analog to Digital Converter (ADC) as well as an 8-bit Pulse Width Modulation (PWM) capability. In that regard, 490 Hz PWM signals can be generated and used for precise temperature control of the thermal block 108 and lid 142. At such high frequency, a pulsed voltage would be sensed as continuous DC source by a conductor. In addition, power MOSFETs were used to control the electrical current flow through each conductor.

Temperatures of the thermal block 108 and lid 142 are measured by acquiring resistances of embedded thermistors 144 in the thermal block 108 and lid 142. For a heater with known conductor material, the resistance as function of temperature is given as,

R(T)=T(T₀)[1+αΔT], (1)

### Where,

R(T)=Conductor resistance at temperature T,
R(T₀)=Conductor resistance at reference temperature T₀
α=Temperature coefficient of resistance for conductor material
T=Conductor temperature in degrees Celsius
T₀=Reference temperature at which a is specified
ΔT=Temperature difference between Conductor temperature and reference temperature

Temperature can be derived from the previous expression for a measured resistance value. A custom Printed Circuit Board (PCB) holds the main electronic components and the whole system fits in a 3D printed enclosure. The overall dimensions are 21 cm×16 cm×20 cm and the unit weighs 500 g.

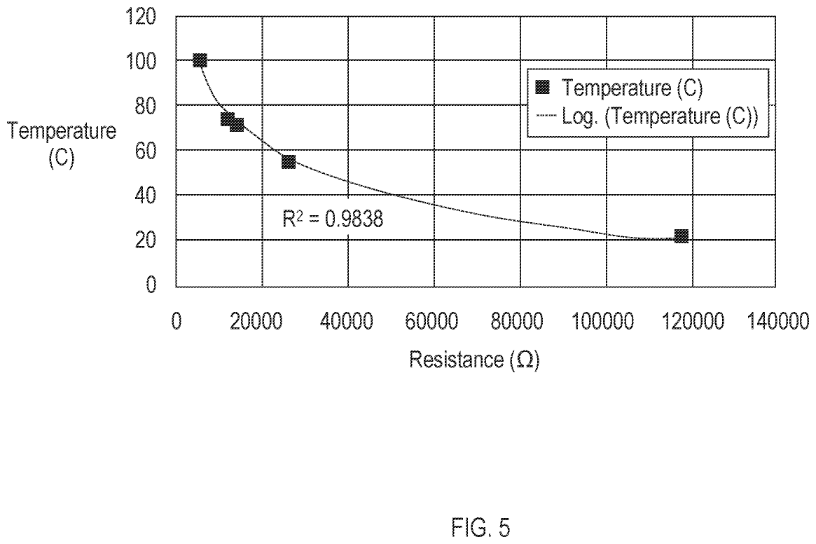

A resistance-temperature curve was generated for the metallic thermal block 173 that was harvested from GeneAmp PCR System 2400 and the data was used to build a look-up table. The resistance was recorded using a bench-top ohmmeter and its corresponding temperature value was acquired with an infrared thermometer. The curve of the temperature as function of resistance has a logarithmic trend with a coefficient of correlation of 0.98, as shown in FIG. 5.

The equation of the resistance-temperature characteristics curve was obtained as,

T=−25.31 ln(R)314.68 (2)

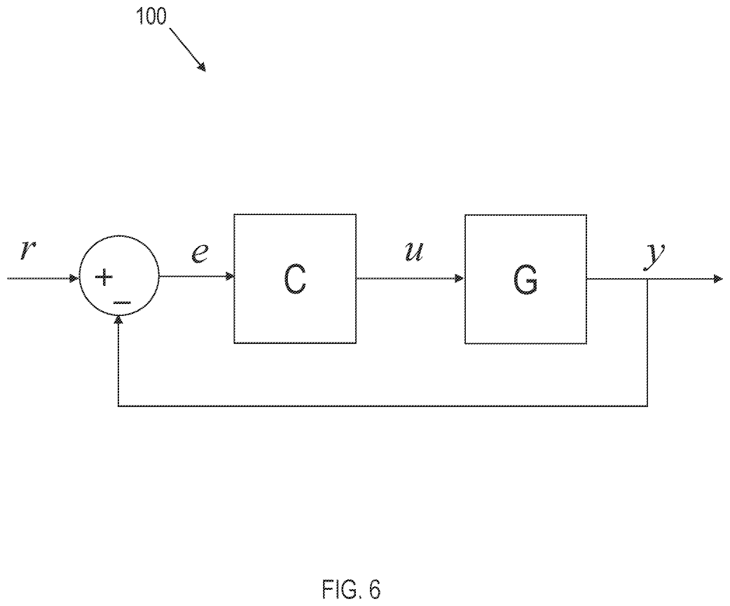

PCR thermal cycling requires precise temperature control to achieve optimal results. In that regard, a novel Proportional—Integral—Derivative (PID) scheme is used as the thermal control system. Because the system could be characterized as not “well-behaved” due to the extended delay in reaction time, regular tuning techniques such as Ziegler—Nichols and Cohen—Coon are not suitable. While using traditional PID control (see FIG. 6) for a PCR reaction would imply switching between temperature setpoints and making the system react accordingly, using such controllers on a highly delayed system would fail to meet the typically 30 seconds (or less) PCR step requirement because of an elongated settling time. Thus, a new method called the Progressive Selective PID Controller (PSPC), overcome the time delay limitation was developed. The PSPC offers a low-cost, compact, and portable PCR system 100, that is fully customizable and autonomous via input interface (e.g., keypad, push buttons, graphical user interface, etc.) 146 and output interfaces (LCDs, LEDs) 148. The user can define the number of PCR steps, the temperature at each step, and the PCR run cycles. The PSPC incorporates fluorescence capability not only for DNA amplicon detection but also for quantification (real-time PCR). Quantification is used to determine an initial amplicon concentration in the PCR solution, which will be useful for food and water quality assessment. PCR status and fluorescence measurement results are displayed on LCDs. The system supports networking capability for data transmission, with an automatically generated HTML interface sent to commonly wireless fidelity (WiFi) connected devices such as phones, computers, and tablets. Embodiments of the system are powered from 120 VAC outlets.

Numerous embodiments of the system 100 are fully built from low-cost components and 3D printed parts. For example, an ATmega2560 is used as microcontroller 140 for I/O management and temperature control. The optical component 104 of the system is 3D printed and includes a spectrometer (e.g., AS7341 10-channel spectrometer) for fluorescence measurement. The system 100 was designed at an overall cost of about $350.

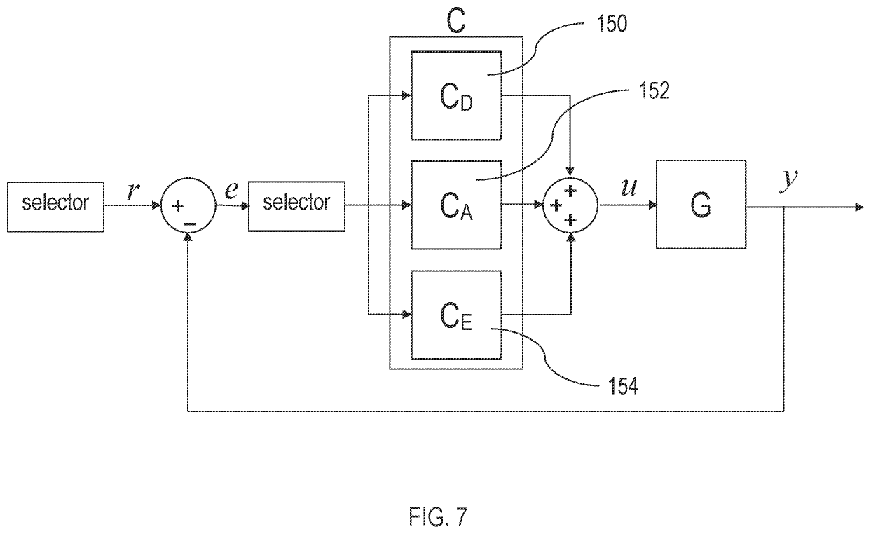

Turning to FIG. 7, in embodiments the disclosed system 100 and method, three PID controllers 150, 152, 154 are executed based on a selector command. The PID controllers control the denaturation phase, the annealing phase, and the extension phase, respectively, for temperature control. The control variable u(t) of each controller is saved in memory and progressively updated as the process continues, which prevents loop recalculation of PID values and allows continuous fine tuning. Therefore, the system 100 becomes more precise over time. The initial controller setting is done using a heuristic approach, depending on the type of material used. The system 100 can be characterized as a second degree PID control system. In regular control, the PID controller can be modeled as,

$$u ( t ) = {K}_{p} e ( t ) + {K}_{I} {\int }_{0}^{t} e ( t ) dt + {K}_{D} \frac{de ( t )}{dt} , \qquad ( 3 )$$

The transfer controller transfer function can be approximated as,

$$C ( s ) = [ \begin{matrix}{K}_{p} & {K}_{I} & {K}_{D}\end{matrix} ] [ \begin{matrix}1 \\ \frac{1}{S} \\ \frac{S}{τ S + 1}\end{matrix} ] \qquad ( 4 )$$

By defining

$${θ}^{T} = [ \begin{matrix}{K}_{p} & {K}_{I} & {K}_{D}\end{matrix} ] , \qquad ( 5 )$$

$${∅}_{1} = [ \begin{matrix}1 \\ \frac{1}{S} \\ \frac{S}{τ S + 1}\end{matrix} ]$$

The equation (2) above can be rewritten as,

C(s)=↓^{T}ø₁ (6)

With the improved PID controller scheme disclosed herein, the controller transfer function is redefined as,

C(s)=(SLR==D)(θ_{D}^{T}ø1)+(SLR==A)(θ_{A}^{T}ø1)+(SLR==E)(θ_{E}^{T}ø1) (7)

### With,

SLR=selector value, D=Denaturation step, A=Annealing step, E=Extension step, θₙ^{T}=PID gains at specified selector value.

Photodetection Procedure

Fluorescence measurements can be performed during thermal cycling after pausing the process at one its stages and removing the control PCR tube from the heating block and placing it into the optical system. Performing fluorescence measurement during the extension stage is optimal as the temperature is low enough to prevent evaporation inside the tube after removing the heated lid and the temperature is suitable to further allow new DNA strands formations. Prolonged exposure to an excitation source (e.g., an LED) has adverse effects on the fluorescence potency of the fluorophore used for real-time PCR monitoring. This effect, known as photobleaching, is intensified by sub-optimal irradiance. Embodiments of the optical system discussed herein has a controlled light emission of three seconds and uses optimal light intensity to reduce photobleaching effects. The various fluorophores used in PCR have different excitation wavelengths which accordingly determines the required excitation LED.

Primers with longer amplicon size tend to yield less amplification efficiency because there is need for more time to complete DNA elongation; therefore such primers reach the reaction plateau at later cycles (high CT). Using that property, it was determined experimentally that with primers of product size between three-hundred and six-hundred base pairs (bp), and a PCR protocol of three steps and forty cycles, starting point amplicons with concentrations at different order of magnitudes yield final DNA concentrations at a gradient. This protocol is used to quantify listeria concentration in water by applying fluorescence on the PCR final products.

System Automation and Versatility

As mentioned previously, PCR reaction settings largely depend on the type of DNA and expected outcome. Using fixed temperatures in thermal cycling would not work well for all assays and must be tailored accordingly. The PSPC process was designed to allow user input and custom make protocols. Users can define the number of cycles, number of steps for each cycle, the step temperatures, step hold times, and the system would automatically adapt without affecting its performance. In addition, that PSPC process was designed to operate autonomously with minimal human interaction during the process. The autonomy and versatility of the system were achieved by developing a C++ program for the ATmega2560 microcontroller that would allow processing the thermal cycling and data communication. An on-board user-friendly interface with keypad, buttons, and display is provided for input/output interaction without the need for external device or computer, even though the user can also remotely access real-time fluorescence data on an external WiFi connected device. The remote data is provided and presented via an automatically generated HTML interface on the device running the PSPC.

Sample Bill of Materials

An embodiment of the disclosed system was designed to be a cost-effective solution with use for resource poor countries. The bill-of-materials cost (see Table 1) for the system was about $340.

TABLE 1
Main parts used in PSPC design

| Parts | Quantity | Cost (Total) |
|---|---|---|
| Peltier Plates | 2 | $63.14 |
| Heat Sink | 1 | $27.96 |
| ESP 8266 Microcontroller | 1 | $6.49 |
| ATMega 2560 Microcontroller | 1 | $15.99 |
| AS7341 Spectrometer | 1 | $15.95 |
| Custom PCB | 1 | $12.62 |
| LCD | 2 | $13.98 |
| 67.2 V 2 A Power Adapter | 1 | $18.99 |
| 30 A Relay | 1 | $12 |
| 12 V 20 A Power Adapter | 1 | $41.99 |
| Fan | 2 | $25.98 |
| Keypad | 1 | $4.50 |
| 1 Kg PLA filament | 1 | $17.99 |
| LED (Light Source) | 1 | $13.99 |
| Optical Filter | 1 | $38.60 |
| Miscellaneous | | $10 |
| Total cost | | $340.17 |

Gel Electrophoresis Analysis

Following the PCR reactions, amplicons quantification was performed using a 1% or 1.2% gel (100 mL Tris-Borate-EDTA (TBE) buffer, 1 gram or 1.2 gram agarose) locally prepared for the experiment, run at 100 volts for 30 minutes, loading 2 μL 6×loading dye and 10 μL PCR sample. The ladder used was 1 kb DNA ladder (New England Biolabs, cat. no. N3232L). The images were captures using UVP Multispectral Imaging System (Biospectrum 500, LM-26, BioChemi 500 Camera f/1.2).

PCR Conditions

The experiments described herein compare the efficiency of the PSPC system versus a commercially available PCR system (Applied Biosystems, model: StepOnePlus). The developed PCR thermal cycling was conducted concurrently on PSPC and the StepOnePlus systems. Fluorescence measurement was compared with commercial device Synergy LX multi-mode reader (BioTek Instruments, model: SLFXA) with Green filter cube loaded (BioTek 1505005, Ex 485/20, M 510, EM 528/20).

Stock primers had a 100 μM concentration and were diluted to a final working stock of 20 μM. All the reaction volumes are 204, on both the PSPC and StepOnePlus. We used 0.2 mL tubes in the PSPC as it allows better adhesion with the heated lid. Tubes used on both PSPC and StepOnePlus were low profile polypropylene tubes, although capillary glass tubes could be used as well. Fresh wild type listeria was streaked from a culture plate. The initial bacterial solution (Tube 1) was performed a serial 1:10 dilution down to 1:10,000,000 (Tube 8) diluted solution using a standard protocol. Tubes 8 and 7 were plated on LB agarose plates for colony count, and count results could be extrapolated for higher concentrations. Amplicon concentrations were mixed with Select MasterMix (Applied Biosystems SYBR, Mfr. No. 4472908) and combined with different primer sets. Table 2 summarizes the target, amplicon size, primer sequence and reaction reagents for gel electrophoresis analysis and fluorescence analysis, respectively. The PCR protocols were identical on both PSPC and StepOnePlus systems, which was as follows: 10 min hot-start at 95° C., followed by 40 cycles of 95° C. (30 sec denaturation), 55° C. (30 sec annealing), 72° C. (60 sec extension), followed by 10 min final elongation at 72° C.

TABLE 2
PCR target, analysis type and reaction components

| Amplicon size | Analysis type | Reagents |
|---|---|---|
| Listeria (115 bp) | Gel electro- | 10 μL Select Master Mix |
| | phoresis | 1 μL F. primer 5′- CAA GCG TTG TCC GGA TTT ATT G -3′ |
| | | 1 μL R. primer 5′- GCA CTC CAG TCT TCC AGT TT -3′ |
| | | 1 μL template |
| | | 7 μL deionized H₂O |
| Listeria (320 bp) | Gel electro- | 10 μL Select Master Mix |
| | phoresis | 1 μL F. primer 5′- GGT GGA GCA TCT GGT TTA ATT C -3′ |
| | | 1 μL R. primer 5′- TTC GCG ACC CTT TGT ACT ATC -3′ |
| | | 1 μL template |
| | | 7 μL deionized H₂O |
| Listeria (629 bp) | Gel electro- | 10 μL Select Master Mix |
| | phoresis | 1 μL F. primer 5′- GTA GCG GTG AAA TGC GTA GA -3′ |
| | | 1 μL R. primer 5′- GCC TAC AAT CCG AAC TGA GAA TA -3′ |
| | | 1 μL template |
| | | 7 μL dionized H₂O |
| Listeria | Gel electro- | 10 μL Select Master Mix |
| (1057 bp) | phoresis | 1 μL F. primer 5′- TGG TTT CGG CTA TCG CTT AC -3′ |
| | | 1 μL R. primer 5′- CTT CGC GAC CCT TTG TAC TAT C -3′ |
| | | 1 μL template |
| | | 7 μL dionized H₂O |
| Listeria | Gel electro- | 10 μL Select Master Mix |
| (1456 BP) | phoresis | 1 μL F. primer 5′- CGA ACG AAC GGA GGA AGA G -3′ |
| | | 1 μL R. primer 5′- GGC TAC CTT GTT ACG ACT TCA -3′ |
| | | 1 μL template |
| | | 7 μL dionized H₂O |
| Listeria (320 bp) | Fluorescence | 10 μL Select Master Mix |
| | | 1 μL F. primer 5′- GGT GGA GCA TCT GGT TTA ATT C -3′ |
| | | 1 μL R. primer 5′- TTC GCG ACC CTT TGT ACT ATC -3′ |
| | | 8 μL template (8 μL dionized H₂O for negative control) |
| Listeria (629 bp) | Fluorescence | 10 μL Select Master Mix |
| | | 1 μL F. primer 5′- GTA GCG GTG AAA TGC GTA GA -3′ |
| | | 1 μL R. primer 5′- GCC TAC AAT CCG AAC TGA GAA TA -3′ |
| | | 8 μL template (8 μL dionized H₂O for negative control) |

Temperature Curve Analysis

The heating and cooling speed recorded for the PSPC was comparable to the StepOnePlus system. With the thermal block fully loaded (24 tubes), we recorded an average of 40 seconds to cool down for 95° C. to 55° C., 9 seconds to heat up from 55° C. to 72° C. and 13 seconds from 72° C. to 95° C. Table 3 summarizes the thermal transition rate between PCR steps at the preset temperatures. For a 40-cycle reaction, it took an average of 2 hours 23 min on PSPC versus 2 hours 10 min on StepOnePlus system. Table 3 summarizes the heating and cooling rates of the system.

TABLE 3
Temperature rates of change during cycles

| Stage | Transition rate |
|---|---|
| 95° C.-55° C. | −1° C./s |
| 55° C.-72° C. | 1.89° C./s |
| 72° C.-95° C. | 1.77° C./s |

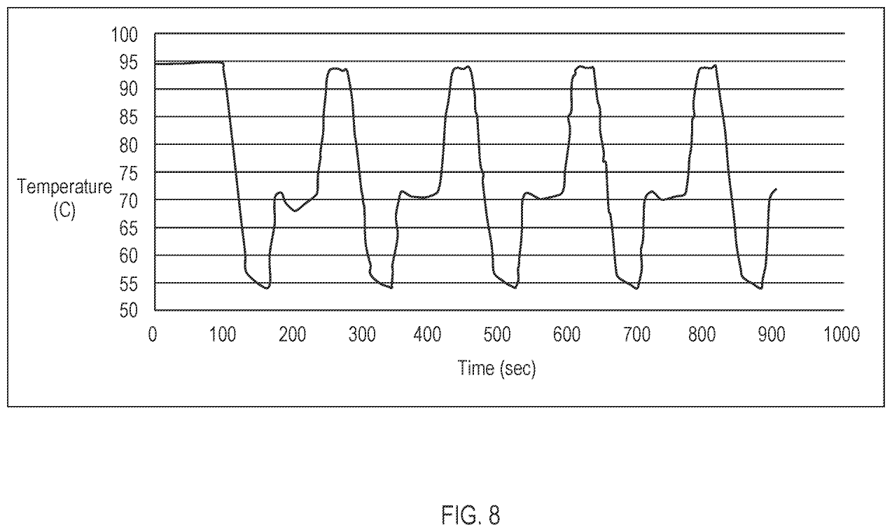

With the selective progressive controller, the temperature curve shows low slope and reduced fluctuations at each temperature step, which is appropriate for temperature selective applications. FIG. 8 shows the temperature curve recorded for the PSPC at 95° C.-55° C.-72° C. cycle.

The temperature curve shows how the system improves over time in maintaining a fixed temperature setpoint. This is the result of the progressive selective control.

Colony PCR Amplification for Listeria Detection

We used PSPC for listeria detection by amplifying DNA from listeria colony as described in the PCR conditions. The total runtime was 2 hours and 23 minutes for PSPC for 40 cycles (30 sec, 30 sec, 60 sec) and 10 min hot-start and 10 min final elongation. Because the heating block is not fully enclosed, a little condensation and evaporation could be observed in the exposed area of tubes, but it does not adversely effect the reaction outcome. The PCR result shows that the right amplicon sizes were amplified, and results are comparable on both systems. Even in instances such as 629 bp product size, the PSPC produced a brighter gel signal compared to StepOnePlus system

Longer product size amplicon amplification was not successful on both systems as the process would require a longer elongation time than the one used in the protocol. Nevertheless, the other results were similar on both systems

Bacterial Count Using Fluorescence

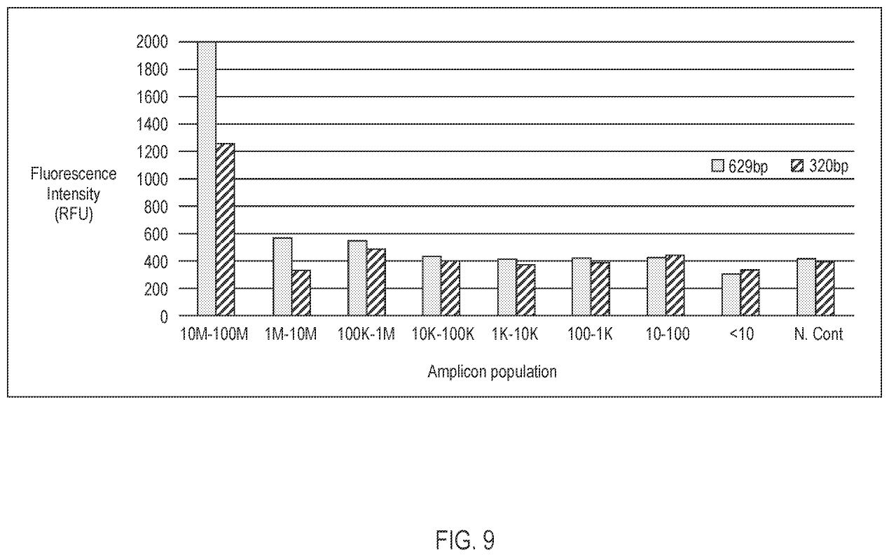

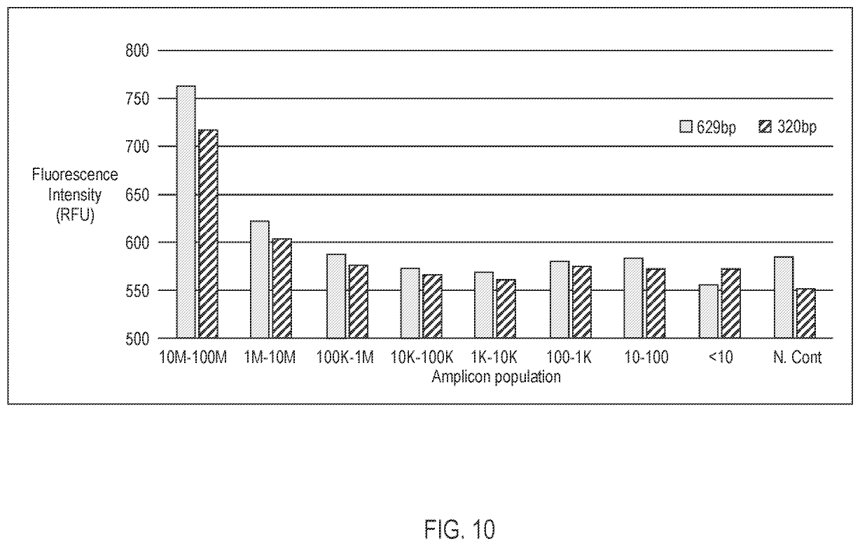

PSPC was used to estimate listeria amplicon population in water, using the prementioned PCR protocol. A two second integration time was used on the sensor for fluorescence measurement. Colony count shows an estimate of less than 10 CFU per PCR tube for 1/10,000,000 dilution. FIGS. 9 and 10 show the summary of the fluorescence level as function of amplicon population on StepOnePlus and PSPC systems. The parallel operation on StepOnePlus and PSPC systems showed similar level of sensitivity. Fluorescence signal recordings show a gradient from tubes 1 to tube 4 (1/1 to 1/1000 dilution) in both settings. Below that threshold, the detection no longer follows a gradient pattern

The experiment did not perform any type of bacteria preservation method which could explain a random decay of amplicon population at lower concentrations. Also, the negative control showed higher fluorescence level as compared to some lower bacteria concentrations. The present experiment did not use a nuclease free water for negative control that could yield a more accurate result.

### CONCLUSIONS

We described the design, development and testing of a portable, low-cost, and real time PCR system that can be used in emergency health crises and point-of-care diagnostics. In addition, the system can be utilized for food and water quality assessment. The described PCR system incorporated real-time reaction monitoring using fluorescence and eliminated the need of gel electrophoresis for reaction analysis, further decreasing the need of multiple reagents, reducing sample testing cost, and reducing sample analysis time. The bill of materials cost of the described system was approximately $340. The described PCR system utilized a novel progressive selective proportional—integral— derivative controller that helps in improving system accuracy and reducing sample analysis time. The PCR system is versatile and provided a clinically relevant performance and can be used with a wide range of DNA samples

Miscellaneous

With the versatility and low cost of the PSPC, the PSPC may be used in medical settings such as hospital, clinics and other healthcare facilities for various medical diagnostics such as COVID-19. For low-income areas of the world that might require a mobile clinic, this device will fit in appropriately. Households, especially those using private drinking water wells, can employ the PSPC for frequent water and food quality check. Industries and private companies can make use of the system for the same intent. Law enforcement agencies can use the PSPC for DNA analysis, anywhere PCR protocol is usable.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable storage medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM), Flash memory, an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer storage medium does not include propagating signals.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Network using an Network Service Provider).

Aspects of the present disclosure are described herein with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the invention in the form disclosed. Many modifications and variations will be apparent to those of ordinary skill in the art without departing from the scope and spirit of the invention. Aspects of the disclosure were chosen and described in order to best explain the principles of the invention and the practical application, and to enable others of ordinary skill in the art to understand the invention for various embodiments with various modifications as are suited to the particular use contemplated.

What is claimed is:

1. A Polymerase Chain Reaction (PCR) system for detecting pathogens in a sample, the system comprising:
a thermal cycler that controls a temperature of the system;
a control system that controls the thermal cycler;
an optical system and readout that displays results of a test of the sample for pathogens; and
a network device that conveys the results of the test of the sample for pathogens.

2. The PCR system of claim 1, wherein the thermal cycler comprises:
a denaturation controller that controls temperature of a denaturation phase;
an annealing controller that controls temperature of an annealing phase; and
an extension controller that controls temperature of an extension phase.

3. The PCR system of claim 2, wherein:
the denaturation controller is a proportional-integral-derivative (PID) controller;
the annealing controller is a proportional-integral-derivative (PID) controller; and
the extension controller is a proportional-integral-derivative (PID) controller.

4. The PCR system of claim 1 further comprising:
an input interface that accepts input from a user, wherein the input indicates a number of PCR steps, a temperature for each PCR step, and a number of PCR run cycles;
wherein the control system controls the thermal cycler based on the input received via the input interface.

5. The PCR system of claim 1, wherein the optical system comprises:
a light-emitting diode (LED) as an excitation source;
a receptacle for holding a PCR tube;
an optical filter; and
a chip-scale spectrometer.

6. The PCR system of claim 5, wherein the chip-scale spectrometer is a 10-channel spectrometer.

7. The PCR System of claim 5, wherein light from the excitation source travels from underneath the receptacle and emitted light travels at a right angle from an excitation spectrum to minimize background noise.

8. The PCR system of claim 5, wherein the optical system further comprises an orange optical filter with a 546 nanometer center wavelength.

9. The PCR system of claim 5, wherein the spectrometer includes a light sensor that detects center wavelengths at 415 nanometers (nm), 445 nm, 480 nm, 515 nm, 555 nm, 590 nm, 630 nm, and 680 nm.

10. The PCR system of claim 1, wherein the thermal cycler comprises:
a heated lid;
a thermal block;
a thermoelectric cooler;
a heat sink; and
a fan.

11. The PCR system of claim 10, wherein the thermal block includes two heaters.
